(19) United States
(12) Patent Application Publication
(10) Pub. No.: US 2021/0187080 A1
(43) Pub. Date: Jun. 24, 2021

(54) PROCESS OF OBTAINING NATIVE TYPE II COLLAGEN OF AVIAN ORIGIN

(71) Applicant: OLNATURA S.A. DE C.V., Jiutepec, Morelos (MX)

(72) Inventor: Alonso ARANDA KILIAN, Ciudad de Mexico (MX)

(73) Assignee: OLNATURA S.A. DE C.V., Jiutepec, Morelos (MX)

(21) Appl. No.: 16/091,322

(22) PCT Filed: Mar. 15, 2018
(86) PCT No.: PCT/MX2018/000020

§ 371 Date: Oct. 4, 2018

(30) Foreign Application Priority Data
Jun. 13, 2017 (MX) MX/A/2017/007745

(51) Int. Cl. A61K 38/39; A61K 35/57

(52) U.S. Cl. CPC A61K 38/39; A61K 35/57

(57) ABSTRACT

The invention relates to a process for obtaining native type II collagen from chicken cartilage useful for preventing or alleviating symptoms of rheumatoid arthritis in humans and mammals in general. The process details the features to obtain said material, its sanitization, drying and grinding, in order to be used in an innocuous way in food supplements. The process generates a microbiologically innocuous pathogen-free product, that can be dosed without risks for its consumption in formulations.

### BRIEF DESCRIPTION OF THE INVENTION

### Technical Field of the Invention

The invention relates to a process for obtaining native type II collagen with the appropriate microbiological quality from chicken cartilage useful for preventing or alleviating symptoms of rheumatoid arthritis in humans and mammals in general.

### BACKGROUND OF THE INVENTION

Collagen works as a lubricant in the joints, avoiding direct friction between two bones. As the disease progresses, and the amount of collagen in a joint is reduced, the bones begin to rub and wear.

The literature notes that a relevant factor in the pathogenesis of rheumatoid arthritis and osteoarthritis is the organism's autoimmune response.

The apparent origin of this problem is the presence of the Epstein-Barr virus, which has an amino acid sequence identical to that of human type II collagen. When the organism generates the antibodies to attack this virus, they also attack their own collagen II. Chicken type II collagen shares some antigenic regions with the same collagen in the human being.

The effect of administering type II collagen activates a process known as oral tolerization. This tolerization refers to the observation that when a protein is administered orally, immunization with the protein leading to a state of systemic hyporesponsiveness or de-sensitization to the immunological factor is generated.

Antibodies to this collagen have a role in the aforementioned pathogenesis, so that its administration can lead to the induction of immune tolerance to this collagen.

Chicken cartilage contains, among other types of collagen, type II collagen, in its native form, that is, with its quaternary structure in the form of a helix.

Chicken cartilage, specifically the sternum or keel thereof, by the presence of this type of collagen, results in an adequate source of the material for its use for these purposes.

Several studies have identified the beneficial potential of the use of this chicken type II collagen, administered orally, to alleviate discomfort associated with the aforementioned condition.

There are several publications that refer to this type of effect on individuals with rheumatoid arthritis and osteoarthritis.

There are several methodologies to process chicken cartilage in the literature, many of them with the use of sodium hypochlorite, others with hydrogen peroxide and others with cartilage radiation (citations: 1,2). Cartilage is processed in various methodologies, including the use of cartilage itself, the same cartilage suspended in liquids with various ingredients, or processed, dry and ground cartilage (citations: 3,4).

The processes referred to herein generate a product within microbiological specifications, but the final product remains with an intermediate or near-high microbial load regarding the general specifications, a load that may undergo alterations over time, leading the product to the risk of falling outside the specifications. The process described herein makes adjustments to the sanitization conditions, through a process with greater concentration of the oxidizing agent and a longer exposure time, to seek that the finished product is with a lower microbial load, which reduces the risks of falling out of specification over time. This is particularly important because given the animal origin of the product, there may be risks in that regard. The resulting process may be a bit longer, but it gives greater confidence in the microbiological quality of the finished product.

### SUMMARY OF THE INVENTION

The process of the present invention involves obtaining sanitized, dried and ground cartilage for use as a supplement to lighten discomforts on individuals with rheumatoid arthritis and osteoarthritis.

It is important to note that hydrolyzed collagen is commercially used for multiple uses, such as cosmetics. The product obtained from this technique is not considered for these applications.

The inventors identified that only a powerful oxidant is necessary, as is the case of hydrogen peroxide, in higher concentrations, and with prolonged times to those known in the state of the art, but without generating vapors harmful to the health of the person responsible for the process, to comply with the microbiological specifications of the product as an input for food supplements.

The process of the present invention ensures the total elimination of any pathogenic microorganism, and reduces the microbial count of aerobic mesophiles and of fungi and yeasts, to levels well below what is specified by the corresponding standards, to ensure that this is not a health risk of those who consume it.

Other techniques that seek to obtain a dry product, use lower concentrations of hydrogen peroxide and less exposure time. In the case of the process of the present invention, the advantage of using stronger conditions allows us to obtain a product consistently within microbiological specifications, without affecting the proteins, because it is the oxidizing agent that does not cause damage to the protein itself, even in greater concentrations. A relevant factor of the process of the present invention is that it was worked with a maximum concentration of 4.5%, because above this value, the solution generates vapors harmful to the health of the personnel who process it. The appearance of the finished product itself, already dry and ground, is a white to beige powder, which facilitates its mixing to make the final presentation for consumption, without the presence of speckles or tone variants, as the excipients frequently used in the industry are white to beige tones too.

With the concentrations, times and temperatures of the present process, a product can be obtained within microbiological specifications, without subjecting the protein to conditions that could cause a relevant degradation thereof. For these purposes, it is necessary to dry said protein with a relatively low temperature, of 40° C. or less, in order to avoid damage to it.

### DETAILED DESCRIPTION OF THE INVENTION

The sternum of a chicken obtained from a processing facility approved by the sanitary authorities, is a hygienic source of collagens, particularly of native type II collagen.

It is very important to maintain the cold chain in the handling of this input, from its supply to its processing. This is due to the presence of proteins such as collagen, which are sensitive to temperature, and the risk of microbiological contamination that occurs in any input of animal origin with high temperatures.

From the cut of the sternum of the chicken, its storage, transport and processing, until the humidity of the material is reduced, it is necessary that the temperature of the input does not exceed 4° C.

This input is hydrated, washed and previously cut to remove impurities from the plant process, to obtain said meat-free sternum and other impurities. It is important to use purified water that is within microbiological specifications for the entire process.

If the cartilage is not immediately processed, it should be safeguarded under refrigeration, at temperatures no higher than 4° C.

The cartilage is subsequently sanitized with a sanitizing agent, in this case hydrogen peroxide, in concentrations ranging from 3.5% to 4.5%, and with an exposure time of 2 to 4 hours. The use of hydrogen peroxide was made after testing the use of sodium hypochlorite, and finding greater effectiveness in hydrogen peroxide. At all times, a single sanitizing agent was looked for to simplify the operation and avoid risks of any kind. The use of radiation was not tested to avoid risking the organoleptic quality of the product. This in order to ensure that any pathogen is completely eliminated, and that the general counts (aerobic mesophilic, fungi and yeast), are lower than those specified for this type of product. Softer conditions (lower concentration and/or shorter time) or the use of other substitute sanitizers of hydrogen peroxide, will result in inconsistency in the presence or absence of some pathogens, and variable amounts of aerobic mesophiles, which may lead to a product very close to the specification limit (1,000 CFU/g of aerobic mesophiles, 100 CFU/g of fungi and yeasts, absence of pathogenic microorganisms such as E. Coli, Salmonella, Staphylococcus aureus, Pseudomonas aeruginosa, with the corresponding risk.

The sanitization is carried out in sanitary containers, preferably stainless steel, preferably type 316. It should be ensured that all the keels are submerged in the sanitizing solution, to achieve the expected effect.

The process temperature of said sanitization should be lower than 30° C., to avoid any damage to collagen.

Once the sternum has been sanitized, it is rinsed with purified water, in a proportion of 2 liters of purified water per kg of keels. This is done for 15 minutes, and it is repeated three times, draining the water in each case. If the process is not continued at this time, the sanitized keel should be stored under refrigeration, at a temperature not higher than 4° C. for further processing.

The keels are dried in a tray dryer, under two variants: at 40° C., in an atmospheric operation (without application of vacuum in drying), or at 15° C. or less with a vacuum of 1.6 kPa (16 mbar), until reaching a humidity less than 5.0%.

Temperatures are essential, to ensure not to damage collagen, which begins to suffer degradation from 42° C.

Drying below 5.0% humidity ensures that the finished product can be stored for long periods of time, without affecting the quality thereof. When storing a product with humidity levels close to or greater than 10%, the water activity is higher and there is a risk of microbial and fungal contamination mainly, which would modify its appearance and its feasibility of use for human consumption.

The dry product is ground or pulverized to a particle size suitable for the type of presentation that is going to be required (capsules, tablets, etc.). During grinding or spraying, it is important to watch that the powder does not heat up above 35° C., to avoid damage to collagen.

### IMPLEMENTATION EXAMPLES

Ex. 1—chicken keel is taken from a certified processing facility by authorities, which should be in refrigeration. This keel is hydrated with 5 liters of purified water for each kg of keel, washed in that water and the excesses like meat and others are cut. It is finely cut and sanitized with 3.5% hydrogen peroxide for 2 hours at a temperature no higher than 30° C. All the sanitizations mentioned in the examples are carried out at 30°. When concluding this stage, it is rinsed with water (2 liters of water per kg of keel), for 15 minutes and the operation is repeated three times, discarding the resulting liquid. This product is loaded into a tray dryer and dried at 40° C., the necessary time to lower the humidity of the finished product below 5.0%. The obtained product, is milled or pulverized to an appropriate particle size for the use it will receive, either in tablets or in capsules.

Ex. 2—the process is carried out under the same conditions of example 1, but 4.0% hydrogen peroxide is used for the same time.

Ex. 3—the process is carried out under the same conditions as in Example 1, but with 4.5% hydrogen peroxide.

Ex. 4—the process is carried out under the same conditions of example 1, but is sanitized for 3 hours.

Ex. 5—the process is carried out under the same conditions of example 4, but 4.0% hydrogen peroxide is applied

Ex. 6—the process is carried out under the same conditions of example 4, but hydrogen peroxide is applied at 4.5%

Ex. 7—the process is carried out under the same conditions of example 1, but is sanitized for 4 hours.

Ex. 8—the process is carried out under the same conditions of example 7, but hydrogen peroxide is applied at 4.0%

Ex. 9—the process is carried out under the same conditions of example 7, but hydrogen peroxide is applied at 4.5%

Ex. 10—the product of example 1 is dried in trays at a temperature of 15° C., applying a vacuum of at least 1.6 kPa (16 mbar), until it reaches humidity below 5.0%.

Ex. 11—comparative example: keel sanitation with 3.0% hydrogen peroxide for 20 minutes, use the product without drying, only finely cut and administered to patients. Conditions described in U.S. Pat. No. 5,750,144A.

Ex. 12, 13—Tests with 3% peroxide and exposure times of one and two hours (insufficient result).

Microbiological measurements were carried out in the cited examples, including samples generated according to a technique published in other patents (lower concentrations of sanitizing agent), in order to confirm that the microbiological quality of the product is improved with the technique proposed herein. Common parameters were measured, such as the presence of aerobic mesophiles measured as colony forming units—CFU/gr, fungi and yeast (CFU/g) and the absence or presence of pathogens such as coliforms, Staphylococcus aureus, Pseudomonas aeuroginosa and Salmonella spp.

| | Mesophiles | Fungi and yeast | |
|---|---|---|---|
| | CFU/gr | CFU/gr | Pathogens |
| Ex. | 1,000 | 100 | absence |
| 1 | 75 | 60 | 0 |
| 2 | 30 | 45 | 0 |
| 3 | 60 | 30 | 0 |
| 4 | 45 | 75 | 0 |
| 5 | 30 | 15 | 0 |
| 6 | 30 | 15 | 0 |
| 7 | 45 | 60 | 0 |
| 8 | 30 | 15 | 0 |
| 9 | 30 | 30 | 0 |
| 10 | 105 | 45 | 0 |
| 11 | 1,545 | 690 | 45 E. Coli |
| 12 | 895 | 60 | 30 E. Coli |
| 13 | 425 | 45 | 0 |

### BIBLIOGRAPHIC REFERENCES

1. U.S. Pat. No. 5,529,786A

2. U.S. Pat. No. 5,645,851A

3. U.S. Pat. No. 5,750,144A

4. U.S. Pat. No. 7,846,487B2

5. Pat. W01997037643A1

6. Science 1993; 2611:1727-1730.

7. Clin.Prac.Alt.Med. 2001; V2, No4, 254-259

8. Arthritis Rheum. 1998; 41: 290-297

1. A process for obtaining native type II collagen from the chicken keel, comprising:
a) obtaining the cartilage from the chicken keel;
b) sanitizing the cartilage with hydrogen peroxide at a concentration between 3.5 to 4.5% and at a temperature below 30° C.;
c) rinsing the cartilage with purified water;
d) drying the cartilage at a temperature equal to or lower than 40° C. until reaching a humidity lower than 5.0%.

2. The process according to claim 1, wherein the chicken sternum comes from a cold chain not higher than 4° C.

3. The process according to claim 1, wherein the sanitization is carried out for 2 to 4 hours.

4. The process according to claim 1, wherein the drying step is carried out under atmospheric conditions.

5. The process according to claim 1, wherein the drying step is carried out at 15° C. or less with a vacuum of 1.6 kPa (16 mbar).

6. The process according to claim 1, wherein it additionally comprises a grinding or pulverizing step at less than 35° C. after the drying step.

7. The process according to claim 2, wherein the sanitization is carried out for 2 to 4 hours.
